SEQUENCE LISTING (11 sequences)

SEQ ID NO: 1 - DNA, 1435 bp, Lactobacillus paracasei
agtcgaacgagttctcgttgatgatcggtgcttgcaccgagattcaacatggaacgagtggcggacgggtgagtaacacgtgggtaacctgcccttaagtgggggataacatttggaaacagatgctaataccgcatagatccaagaaccgcatggttcttggctgaaagatggcgtaagctatcgcttttggatggacccgcggcgtattagagttggtgaggtaatggctcaccaaggcgatgatacgtagccgaactgagaggttgatcggccacattgggactgagacacggcccaaactcctacgggaggcagcagtagggaatcttccacaatggacgcaagtctgatggagcaacgccgcgtgagtgaagaaggctttcgggtcgtaaaactctgttgttggagaagaatggtcggcaagtaactgttgtcggcgtgacggtatccaaccagaaagccacggctaactacgtgccagcagccgcggtaatacgtaggtggcaagcgttatccggatttattgggcgtaaagcgagcgcaggcggttttttaagtctgatgtgaaagccctcggcttaaccgaggaagcgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagcatgggtagcgaacaggattagataccctggtagtccatgccgtaaacgatgaatgctaggtgttggagggtttccgcccttcagtgccgcagctaacgcattaagcattccgcctggggagtacgaccgcaaggttgaaactcaaaggaattgacgggggcccgcacaagcggtggagcatgtggtttaattcgaagcaacgcgaagaaccttaccaggtcttgacatcttttgatcacctgagagatcaggtttccccttcgggggcaaaatgacaggtggtgcatggttgtcgtcagctcgtgtcgtgagatgttgggttaagtcccgcaacgagcgcaacccttatgactagttgccagcatttagttgggcactctagtaagactgccggtgacaaaccggaggaaggtggggatgacgtcaaatcatcatgccccttatgacctgggctacacacgtgctacaatggatggtacaacgagttgcgagaccgcgaggtcaagctaatctcttaaagccattctcagttcggactgtaggctgcaactcgcctacacgaagtcggaatcgctagtaatcgcggatcagcacgccgcggtgaatacgttcccgggccttgtacacaccgcccgtcacaccatgagagtttgtaacacccgaagccggtggcgtaacccttttagggagcgagccgtctaa

SEQ ID NO: 2 - DNA, 20 bp, Artificial Sequence
  IL-6 Forward
tccagttgccttcttgggac

SEQ ID NO: 3 - DNA, 22 bp, Artificial Sequence
  IL-6 Reverse
gtgtaattaagcctccgacttg

SEQ ID NO: 4 - DNA, 24 bp, Artificial Sequence
  TNF-alpha Forward
ggctttccgaattcactggagcct

SEQ ID NO: 5 - DNA, 29 bp, Artificial Sequence
  TNF-alpha Reverse
ccccggccttccaaataaatacattcata

SEQ ID NO: 6 - DNA, 20 bp, Artificial Sequence
  MIP-1 alpha Forward
caccatatggctcggacacc

SEQ ID NO: 7 - DNA, 21 bp, Artificial Sequence
  MIP-1 alpha Reverse
tcaggaaaatgacacctggct

SEQ ID NO: 8 - DNA, 18 bp, Artificial Sequence
  CXCL1 Forward
ccgaagtcatagccacac

SEQ ID NO: 9 - DNA, 18 bp, Artificial Sequence
  CXCL1 Reverse
gtgccatcagagcagtct

SEQ ID NO: 10 - DNA, 20 bp, Artificial Sequence
  GAPDH Forward
gtcttcctgggcaagcagta

SEQ ID NO: 11 - DNA, 20 bp, Artificial Sequence
  GAPDH Reverse
ctggacagaaaccccacttc

(19) United States
(12) Patent Application Publication
(10) Pub. No.: US 2025/0090605 A1
(43) Pub. Date: Mar. 20, 2025

(54) NOVEL LACTOBACILLUS PARACASEI ATG-E1 STRAIN OR COMPOSITION COMPRISING SAME FOR PREVENTING OR TREATING RESPIRATORY DISEASE

(71) Applicants: Ji Hee KANG, Daejeon (KR); ATOGEN CO., LTD, Daejeon (KR)

(72) Inventors: Ji Hee KANG, Daejeon (KR); Young Sil LEE, Sejong (KR); Dae Young LEE, Daejeon (KR); Sung Hoon IM, Sejong (KR); II Yong JI, Daejeon (KR); Gun Seok PARK, Daejeon (KR); Seung Hyun KO, Sejong-si (KR); Juy I PARK, Daejeon (KR); You Kyung LEE, Daejeon (KR)

(73) Assignees: Ji Hee KANG, Daejeon (KR); ATOGEN CO., LTD, Daejeon (KR)

(21) Appl. No.: 18/580,997

(22) PCT Filed: Jun. 14, 2022
(86) PCT No.: PCT/KR2022/008333

§ 371 Date: Jan. 19, 2024

(30) Foreign Application Priority Data
Jul. 23, 2021 (KR) 10-2021-0097130

(51) Int. Cl. A61K 35/747; A61K 9/00; A61K 9/20; A61P 11/00; A61P 31/06; C12N 1/20; C12R 1/225

(52) U.S. Cl. CPC A61K 35/747; A61K 9/0095; A61K 9/2018; A61K 9/2059; A61K 9/2063; A61P 11/00; A61P 31/06; C12N 1/205; C12R 2001/225

(57) ABSTRACT

A novel strain, Lactobacillus paracasei ATG-E1 (Accession No. KCTC 14245BP), and a composition containing the same for preventing or treating respiratory diseases which are caused by fine particulate matter. The Lactobacillus paracasei ATG-E1 strain reduces the number of immune cells bronchoalveolar and lung tissues and inhibits the expression of inflammatory cytokines such as interleukin-17A (IL-17A), tumor necrosis factor-α (TNF-α), macrophage inflammatory protein 2 (MIP2), C-X-C motif chemokine ligand 1 (CXCL-1), macrophage inflammatory protein-α (MIP-1α) or interleukin-6 (IL-6). Therefore, the Lactobacillus paracasei ATG-E1 strain can be used for a treatment or health functional food for various respiratory diseases such as acute and chronic bronchitis, catarrhal bronchitis, bronchitis obliterans, inflammatory bronchitis, bronchial asthma, atopic asthma, non-atopic asthma, atopic IgE-mediated asthma, allergic asthma, non-allergic asthma, chronic bronchoconstriction, acute bronchoconstriction, chronic obstructive pulmonary disease, bronchial adenoma, pulmonary tuberculosis, emphysema, lung abscess, pulmonary fibrosis, lung cancer, airway cancer, bronchoalveolar cancer, and bronchial cancer.

### TECHNICAL FIELD

The present disclosure relates to a novel strain, Lactobacillus paracasei ATG-E1 (Accession No. KCTC 14245BP), or a composition containing the same for preventing or treating respiratory diseases.

### BACKGROUND ART

Fine dust is particulate matter that is suspended in the air or drifts down to the ground. The fine dust is produced by the burning of fossil fuels such as coal and oil as well as by the combustion in industrial sites and the combustion of vehicle fuels.

The fine dust is categorized by the diameter of the particles, with PM10 referring to particles smaller than 10 μm and PM2.5 referring to particles smaller than 2.5 μm. The fine dust is known to be so fine that it is not filtered out through nasal hair or bronchial mucosa and can enter the alveoli or brain directly when inhaled. Once fine dust enters the body, the cells responsible for immunity are triggered to remove the particulate matter to defend the body, which can lead to an inflammatory response. This inflammatory response in the airways, lungs, cardiovascular system, and brain has been reported to increase the prevalence of asthma, respiratory and pulmonary diseases, and premature mortality. Unlike bacteria, temporary toxic materials, or other substances entering the airways to cause damage to the lungs or respiratory system, fine dust cannot be fought off by the human immune system and cannot be driven out by any means once the fine dust enters the body through the respiratory tract. Furthermore, since it is impossible to know exactly what problems will result from the damage, there is an urgent need for new therapeutics that can prevent, mitigate, treat, or ameliorate the damage to the airways and lungs induced by fine particulate matter, and that can treat respiratory diseases caused thereby.

Lactobacilli, a class of probiotics, live symbiotically in the human digestive system and break down fiber and complex proteins into important nutritional components. Lactobacilli are anaerobic bacteria that break down and use carbohydrates to make lactic acid and thrive in low-oxygen environments. Recently, the prevention and treatment effects of lactobacilli on various diseases have been confirmed, and attempts have been made to develop health functional foods and treatments using the lactobacilli.

While conducting various studies on ways to treat respiratory diseases using Lactobacilli, the inventors confirmed that the new strain of Lactobacillus paracasei ATG-E1 regulates respiratory immunity and suppresses inflammatory responses caused by the toxicity of fine dust, and thus completed the present disclosure.

### DOCUMENTS OF RELATED ART

### Patent Document

Korean Patent No. 10-2165930 (Title of Disclosure: Composition Comprising Lactic Acid Bacteria for Treating Respiratory Diseases or Inflammatory Diseases caused by Stimulation of Fine Dust, Applicant: GC Wellbeing Corporation, Date of Registration: Oct. 7, 2020)

Korean Patent No. 10-2049700 (Title of Disclosure: Composition Comprising Lactobacillus Reuteri ATG-F4 for Preventing or Treating Muscle diseases, Applicant: AtoGEN Corporation, Date of Registration: Nov. 21, 2019)

Korean Patent No. 10-2163551 (Title of Disclosure: Composition Comprising Lactobacillus Plantarum ATG-K2 or ATG-K6 for Preventing and Treating Lipid-related Metabolic Diseases, Applicant: AtoGEN Corporation, Data of Registration: Sep. 29, 2020)

Korean Patent No. 10-1500974 (Title of Disclosure: Lactobacillus Plantarum HAC01 strain with Anti-inflammatory and Metabolic Disease Treatment Effect and Uses Thereof, Applicant: AtoGEN Corporation, Data of Registration: Mar. 4, 2015)

Korean Patent No. 10-1951919 (Title of Disclosure: Novel Lactobacillus Reuteri ATG-F4 Strain with Function of Enhancing Dopamine Secretion and Composition Comprising Same for Preventing or Treating Mental Illness, Applicant: AtoGEN Corporation, Date of Registration: Feb. 19, 2019)

### DISCLOSURE

### Technical Problem

The objective of the present disclosure is to provide a novel strain, Lactobacillus paracasei ATG-E1 (Accession No. KCTC 14245BP), and a composition containing the same for preventing or treating respiratory diseases.

### Technical Solution

The present disclosure relates to Lactobacillus paracasei ATG-E1 strain deposited under an accession number of KCTC 14245BP.

The strain may include a live cell, a dead cell, a culture, a culture fluid containing cells, a culture fluid from which cells have been removed, a concentrate of the culture, or a metabolite isolated from the cell or culture fluid.

The strain may have preventive or therapeutic efficacy for respiratory diseases induced by fine particulate matter.

The respiratory disease may be a disease selected from the group consisting of acute and chronic bronchitis, catarrhal bronchitis, bronchitis obliterans, inflammatory bronchitis, bronchial asthma, atopic asthma, non-atopic asthma, atopic IgE-mediated asthma, allergic asthma, non-allergic asthma, chronic bronchoconstriction, acute bronchoconstriction, chronic obstructive pulmonary disease, bronchial adenoma, pulmonary tuberculosis, emphysema, lung abscess, pulmonary fibrosis, lung cancer, airway cancer, bronchoalveolar cancer, and bronchial cancer.

The strain may have the effect of reducing the number of inflammatory cells and immunoregulatory cells in bronchoalveolar or lung tissue. Therefore, the Lactobacillus paracasei ATG-E1 strain may have the effect of reducing the expression level of an inflammatory cytokine such as interleukin-17A (IL-17A), tumor necrosis factor-α (TNF-α), macrophage inflammatory protein 2 (MIP2), C-X-C motif chemokine ligand 1 (CXCL-1), macrophage inflammatory protein-α (MIP2, MIP-1α) or interleukin-6 (IL-6).

The present disclosure relates to a pharmaceutical composition for treating or ameliorating respiratory diseases, the composition containing the Lactobacillus paracasei ATG-E1 strain.

The present disclosure provides a health functional food or food composition for preventing or ameliorating respiratory diseases, the health functional food or food composition containing the Lactobacillus paracasei ATG-E1 strain.

Hereinafter the present disclosure will be described in detail.

The Lactobacillus paracasei ATG-E1 strain is characterized in that it does not produce biogenic amines of histamine, tyramine, putrecine, and cadaverine.

The Lactobacillus paracasei ATG-E1 strain has acid resistance or bile resistance.

The Lactobacillus paracasei ATG-E1 strain has no resistance against one or more antibiotics selected from the group consisting of ampicillin, vancomycin, gentamicin, kanamycin, streptomycin, clindamycin, erythromycin, tetracycline, and chloramphenicol.

The composition containing the ATG-E1 strain of the present disclosure may contain one or more selected from the group consisting of a live cell of the strain, a killed cell of the strain, a culture of the strain, a culture fluid containing a cell of the strain, a culture fluid from which a cell of the strain has been removed, a concentrate of the culture fluid, and a metabolite isolated from the cell or culture.

The strain of the present disclosure can be cultured in a liquid (broth) or solid (agar) medium of MRS, to a concentration of about 1×10¹⁰ CFU/mL.

The strain is preferably cultured at a temperature in the range of from 35° C. to 40° C. for 8 to 20 hours. The optimum temperature for cultivation is 37° C., the lowest temperature is 15° C., and the highest temperature is 45° C. In addition, the optimum pH for cultivation is 6.0, the minimum pH is 4.0, and the maximum pH is 7.8. The optimal cultivation time is 16 hours, with a minimum cultivation time of 8 hours and a maximum cultivation time of 24 hours.

The present disclosure also provides a pharmaceutical composition for the prevention or treatment of respiratory diseases, in which the pharmaceutical composition contains a novel strain, Lactobacillus paracasei ATG-E1. The novel Lactobacillus paracasei ATG-E1 strain may be contained in an amount of 0.001 to 100 wt % in the pharmaceutical composition of the present disclosure.

The pharmaceutical composition may be formulated and used in the form of oral formulations such as pills, granules, tablets, capsules, suspensions, emulsions, syrups, aerosols, and the like, or in the form of topical formulations, suppositories, and the like, in which the formulations are prepared according to conventional methods. Examples of carriers, excipients, and diluents that can be included in the pharmaceutical composition include lactose, dextrose, sucrose, sorbitol, mannitol, xylitol, erythritol, maltitol, starch, acacia rubber, alginate, gelatin, calcium phosphate, calcium silicate, cellulose, methyl cellulose, microcrystalline cellulose, polyvinylpyrrolidone, water, methylhydroxybenzoate, propylhydroxybenzoate, talc, magnesium stearate, and mineral oil. When formulated, the formulations are prepared using commonly used diluents or excipients such as fillers, extenders, binders, wetting agents, disintegrants, and surfactants. Solid formulations for oral administration include tablets, pills, powders, granules, capsules, and the like. These solid formulations are prepared by adding at least one or more excipients such as starch, calcium carbonate, sucrose, lactose, gelatin, and the like to the composition. Aside from the simple excipients, lubricants such as magnesium stearate, talc, or the like may be additionally used. Liquid formulations for oral administration may include suspensions, solutions, emulsions, and syrups. Aside from a simple diluent such as water or liquid paraffin, the oral formulations may additionally contain various excipients such as wetting agents, sweetening agents, fragrances, preservatives, and the like. Formulations for parenteral administration include sterile aqueous solutions, non-aqueous solutions, suspensions, emulsions, lyophilized preparations, suppositories, and vaginal suppositories. For the non-aqueous solutions and suspensions, propylene glycol, polyethylene glycol, vegetable oils such as olive oil, injectable esters such as ethylolate, and the like may be used. As a base for suppositories, Witepsol, Macrogol, Wwin61, cacao butter, laurin butter, glycerogelatin, and the like may be used.

The dosage of the pharmaceutical composition of the present disclosure will depend on the age, sex, and weight of the subject to be treated, the specific disease or pathological condition to be treated, the severity of the disease or pathological condition, the route of administration, and the determination of the prescriber. Dosing decisions based on these factors may be made depending on the skill of those skilled in the art, and the doses may typically range from about 0.01 mg/kg/day to about 2000 mg/kg/day. More preferred doses range from 1 mg/kg/day to 500 mg/kg/day. The amount can be administered as a single once a day or may be administered as multiple doses. Such dosages should not be construed as limiting the scope of the present disclosure in any respect.

The pharmaceutical composition of the present disclosure can be administered to mammals, such as rats, livestock, and humans, by various routes. The strain of the present disclosure has little toxicity and side effects, so it is an agent that can be safely used for long-term use for prophylactic purposes.

In addition, the present disclosure also provides a health functional food pharmaceutical composition for the prevention or treatment of respiratory diseases, in which the health functional food contains a novel strain, Lactobacillus paracasei ATG-E1. The Lactobacillus paracasei ATG-E1 strain may be contained in an amount of 0.001 to 100 wt % in the health functional food of the present disclosure. The health functional food of the present disclosure is prepared in the form of pills, tablets, capsules, pills, or liquids. The health functional foods to which strain of the present disclosure can be added include, for example, various beverages, meats, sausages, breads, candies, snacks, noodles, ice creams, dairy products, soups, ionic beverages, sodas, alcoholic beverages, chewing gums, teas, and vitamin complexes.

### Advantageous Effects

The present disclosure relates to a novel strain, Lactobacillus paracasei ATG-E1 (Accession No. KCTC 14245BP), and a composition containing the same for preventing or treating respiratory diseases which are caused by fine particulate matter. The Lactobacillus paracasei ATG-E1 strain inhibits the activity of inflammatory cells and various immune cells in bronchoalveolar and lung tissues and inhibits the expression of inflammatory cytokines such as interleukin-17A (IL-17A), tumor necrosis factor-α (TNF-α), macrophage inflammatory protein 2 (MIP2), C-X-C motif chemokine ligand 1 (CXCL-1), macrophage inflammatory protein-α (MIP2, MIP-1a) or interleukin-6 (IL-6). Therefore, the Lactobacillus paracasei ATG-E1 strain can be used for a treatment or health functional food for various respiratory diseases such as acute and chronic bronchitis, catarrhal bronchitis, bronchitis obliterans, inflammatory bronchitis, bronchial asthma, atopic asthma, non-atopic asthma, atopic IgE-mediated asthma, allergic asthma, non-allergic asthma, chronic bronchoconstriction, acute bronchoconstriction, chronic obstructive pulmonary disease, bronchial adenoma, pulmonary tuberculosis, emphysema, lung abscess, pulmonary fibrosis, lung cancer, airway cancer, bronchoalveolar cancer, and bronchial cancer.

### DESCRIPTION OF DRAWINGS

FIG. 1 shows the total cell count and neutrophils cell count in bronchoalveolar lavage fluid derived from an animal model treated with the Lactobacillus paracasei ATG-E1 strain of the present disclosure and fine particulate matter.

FIG. 2 shows the results of determining the protein expression levels of intracellular inflammatory cytokines in bronchoalveolar lavage fluid derived from an animal model treated with the Lactobacillus paracasei ATG-E1 strain of the present disclosure and fine particulate matter.

FIG. 3 shows the results of determining the mRNA expression levels of inflammatory cytokines in lung tissue cells of an animal model treated with the Lactobacillus paracasei ATG-E1 strain of the present disclosure and fine particulate matter.

FIG. 4 shows photographs of stained lung tissue of an animal model treated with the Lactobacillus paracasei ATG-E1 strain of the present disclosure and fine particulate matter.

FIG. 5 is a view showing the 16S rRNA base sequence of the Lactobacillus paracasei ATG-E1 of the present disclosure.

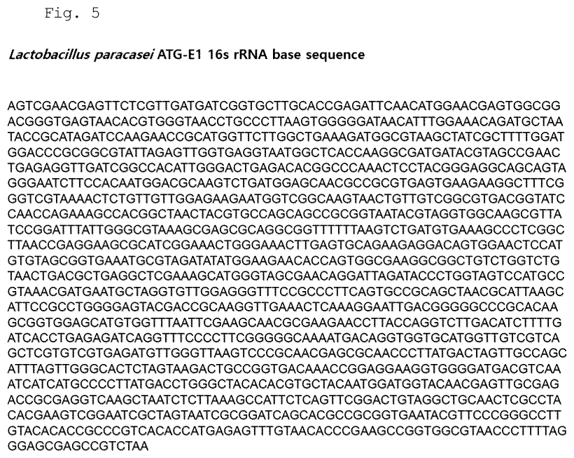

### BEST MODE

Hereinafter, preferred embodiments of the present disclosure will be described in detail. However, the present disclosure is not limited to the embodiments described herein and may be embodied in other forms. Rather, the embodiments are provided so that this disclosure will be thorough and complete and will fully convey the spirit of the disclosure to those skilled in the art.

### Example 1. Isolation and Identification of a Novel Lactic Acid Bacterium, Lactobacillus paracasei ATG-E1

For strain isolation, baby (newborn) feces were obtained. 90 ml of physiological saline (0.85% NaCl/L) was added to an autoclaved bag, and 10 g of the raw sample (baby feces) was added thereto and mixed evenly. 1 ml of the sample mixture was dispensed, serially diluted 10-fold in 9 ml of physiological saline, and smeared on an MRS solid medium three times. Next, the strain was incubated in a 37° C. incubator for at least 48 hours, and the grown strain was analyzed by a catalase test using 0.3% hydrogen peroxide and Gram staining. The bacteria that were catalase-negative, Gram-positive, and bacillus rod-shaped were selected, and the 16S rDNA gene of the selected strain was sequenced. The sequence obtained by 16S rRNA sequencing was compared with the NCBI BLAST database. Since the 16S rRNA sequence of the strain was 99.9% identical to Lactobacillus paracasei, it was determined that the strain belonged to Lactobacillus paracasei in the taxonomic designation.

In addition, the completed whole genome sequence with a size of 3,124,497 bp was obtained using the MinION sequencing platform of Oxford Nanopore Technologies, and compared with the whole genome of the standard strain “Lactobacillus paracasei (ATCC 25302)” by the Average Nucleotide Identity (ANI) analysis. The analysis results showed 98.37% similarity, indicating a difference of 1.63% in the whole genome, meaning that the tested strain was a novel strain (Table 1). Therefore, the raw matter for the present application is named Lactobacillus paracasei ATG-E1, and the strain has been deposited at the Korean Collection for Type Cultures (KCTC) under the accession number “KCTC 14245BP”.

TABLE 1

| Classification | Numerical value |
|---|---|
| ANI value | 98.37% |
| ATG-E1 genome size | 3,124,497 bp |
| Type strain genome size | 2,939,640 bp |

### Example 2. Characterization of a Novel Lactic Acid Bacterium, Lactobacillus paracasei ATG-E1

### Example 2-1. Sugar utilization characteristics

The sugar utilization characteristics of Lactobacillus paracasei ATG-E1 strain isolated according to the present disclosure were determined by using the API 50 CH test (Biomerieux) which was performed according to the instructions described in the manual. As a result, the Lactobacillus paracasei ATG-E1 strain of the present disclosure was observed to have the sugar utilization characteristics shown in Table 2 below.

TABLE 2

| | Use for | | Use for |
|---|---|---|---|
| Carbohydrate | fermentation | Carbohydrate | fermentation |
| Control | − | Esculin ferric | + |
| | | citrate | |
| Glycerol | − | Salicin | + |
| Erythritol | − | D-cellobiose | + |
| D-arabinose | − | D-maltose | + |
| L-arabinose | − | D-lactose | + |
| D-ribose | + | D-melibiose | − |
| D-xylose | − | D-saccharose | + |
| | | (sucrose) | |
| L-xylose | − | D-trehalose | + |
| D-adonitol | + | Inulin | − |
| Methyl-D- | − | D-melezitose | + |
| xylopyranoside | | | |
| D-lactose | + | D-raffinose | − |
| D-glucose | + | Amydon (starch) | + |
| D-fructose | + | Glycogen | − |
| D-manose | + | Xylitol | − |
| L-sorbose | + | Gentiobiose | + |
| L-rhamnose | − | D-turanose | + |
| Dulcitol | − | D-lyxose | − |
| Inositol | − | D-tagatose | + |
| D-manitol | + | D-fucose | |
| D-sorbitol | + | L-fucose | − |
| Methyl-D- | − | D-arabitol | − |
| xylopyranoside | | | |
| Methyl-D- | + | L-arabitol | − |
| xylopyranoside | | | |
| N- | + | Potassium Gluconate | + |
| acetylglucosamine | | | |
| Amygdalin | − | Potassium 2- | + |
| | | KetoGluconate | |
| Arbutin | + | Potassium 5- | − |
| | | KetoGluconate | |

### Example 2-2. Test for Acid Resistance and Bile Resistance

Acid resistance and bile resistance tests of the strain were performed using the simulated stomach duodenum passage (SSDP) method under conditions similar to the human body (stomach and duodenum) environment. The isolated Lactobacillus paracasei ATG-E1 strain was cultured in MRS liquid medium for 18 hours, and then 1 ml of the strain fluid was collected in a tube and centrifuged (3000×g, 5 minutes, 4° C.). The supernatant was discarded, and the cell pellet was washed with physiological saline. The process was repeated twice. The saline was then removed, and the precipitated bacteria were mixed in 10 ml of MRS liquid medium adjusted to pH 3. 1 ml of that suspension was taken, serially diluted 10-fold, smeared on MRS solid medium, and incubated for 24 to 48 hours at 37° C. After incubation at 37° C. for 24 to 48 hours, the number of bacteria produced was counted to determine the initial CFU/ml of the bacteria. The remaining 9 ml was incubated at 37° C. for 1 hour, and then successively mixed with bile acid and 17 ml of duodenal fluid. The bile acid was prepared by mixing 10 g of Ox gall with 100 ml of distilled water and autoclaving the mixture. The duodenal fluid was prepared by mixing duodenum juice, 6.4 g/L of NaHC0₃, 0.239 g/L of KCl, 1.28 g/L of NaCl in distilled water, adjusting the mixture to pH 7.4, and autoclaving the mixture. The survival rate of the bacteria was calculated by comparing the initial CFU/ml to the CFU/ml of the bacteria that survived after 3 hours.

TABLE 3

| | Early | Survival rate (%) | Survival rate (%) |
|---|---|---|---|
| | survival | after one hour at | after two or more |
| | rate (%) | pH 3.0 | hours in 3% Ox gall |
| Lactobacillus | 100 | 73.0 | 51.48 |
| paracasei ATG-E1 | | | |

As confirmed from table 3, it can be seen that the survival rate of Lactobacillus paracasei ATG-E1 is very high under gastric acidic conditions while passing through bile to the small intestine.

### Example 2-3 Antibiotic Resistance Test

The minimum inhibitory concentration (MIC) values of the antibiotics including ampicillin, vanacomycin, gentamicin, kanamycin, streptomycin, clindamycin, erythromycin, tetracycline, and chlorampenicol were determined using nine antibiotic E-test strips (BuoMerieux, France). Briefly, the lactic acid bacteria to be tested were suspended at an OD₆₀₀ absorbance of about 0.8 and smeared onto MRS solid media using a sterilized swab. The solid medium smeared with the lactic acid bacteria was dried for about 3 minutes, and an E-test strip was placed on the medium. The medium was incubated at 37° C. for about 48 hours. Due to the nature of lactic acid bacteria, intrinsic resistance against the aminoglycoside classes such as genetamicin, kanamycin, and streptomycin may be exhibited. Therefore, plate count agar (PCA, Difo Laboratories, USA) or Mueller-Hinton agar (MHA, Difco Laboratories, USA) was used as the test medium for these antibiotics. Guidelines published by the European Food Safety Authority (EFSA) were used to determine the types of antibiotics and the lowest inhibitory concentration that can be considered safe.

TABLE 4

| | AMP | VAN | GEN | KAN | STR | CD | ERY | TET | CM |
|---|---|---|---|---|---|---|---|---|---|
| Lactobacillus paracasei ATG-E1 | 2 | NR | 24 | 48 | 16 | 0.19 | 0.125 | 0.75 | 4 |
| European food safety authority | 4 | NR | 32 | 64 | 64 | 1 | 1 | 4 | 4 |
| (EFSA) | | | | | | | | | |

AMP: ampicillin; VAN: vancomycin, GEN: Gentamicin, KAN: kanamycin; STR: streptomycin; CD: clindamycin; ERY: erythromycin; TET: tetracyclin; CM: chloramphenicol; Cl: ciprofloxacin; NR: not required. (unit: μg/ml)

The results of each assay are shown in Table 4. Table 4 shows the measurement results of the minimum inhibitory concentration (MIC) of Lactobacillus paracasei ATG-E1 (lactic acid bacteria) against major antibiotics. The strain of the present disclosure was found to exhibit a significantly lower level of antibiotic susceptibility than the limit suggested by the EFSA guidelines. This means that the strain “Lactobacillus paracasei ATG-E1” does not pose a risk of exchanging antibiotic resistance genes with other strains. In addition, when the pathogenicity of Lactobacillus paracasei ATG-E1 was predicted using PathogenFinder( ) along with the subsequent whole genome information, no cases were matched with pathogenic families and 480 cases were matched with non-pathogenic families, indicating that Lactobacillus paracasei ATG-E1 is a non-human pathogen and is not likely to be harmful to humans.

### Example 2-4 Biogenic Amine Production Test

An MRS liquid medium added with 1% of amino acid precursors (L-tyrosine disodium salt, L-histidine monohydrochloride monohydrate, L-ornithine monohydrochloride, and L-lysine monohydrochloride) was prepared, and the isolated Lactobacillus paracasei ATG-E1 strain was inoculated on the MRS liquid medium containing the 1% amino acid precursors and subcultured for 5 to passages to activate decarboxylase. Enzyme-activated bacteria were smeared on a decarboxylase medium (tryptone 0.5%, yeast extract 0.5%, cocoon extract 0.5%, sodium chloride 0.5%, glucose 0.25%, Tween-80 0.05%, magnesium sulfate 0.02%, manganese sulfate 0.005%, iron sulfate 0.004%, citrate 0.2%, thiamine 0.001%, K₂PO₄ 0.2%, calcium carbonate 0.01%, pyridoxal-5-phosphate 0.005%, amino acid 1%, bromocresol purple 0.006%, and agar 2% were mixed in distilled water and adjusted to pH 5.3). The bacteria were then cultured at 37° C. for 48 hours to determine the ability to produce biogenic amines by checking whether the color changed to purple.

TABLE 5

| | tyramine | histamine | putrescine | cadaverine |
|---|---|---|---|---|
| Lactobacillus | — | — | — | — |
| paracasei ATG-E1 | | | | |

As shown in Table 5, as a result of analyzing whether ATG-EI strain can produce the biogenic amines including tyramine, histamine, putrescine, and cadaverine from the amino acid precursors including tyrosine, histidine, ornithine, and lysine, the results were confirmed to be negative. That is, the strain of the present disclosure does not have the ability to produce biogenic amines causing a hypersensitivity immune response.

### Example 3. Prevention and Amelioration of Respiratory Damage Caused by Air Pollutants Such as Fine Dust

### Example 3-1 Respiratory-damaged mouse model experiment

There were 8 Balb/c mice (male, 8 weeks old) per group, and all groups except a normal group were injected with a fine dust mixture using the Intra-Nazal-Trachea (INT) injection method described in literatures. The fine dust mixture contained diesel exhaust particles (DEP) (PM10 (Sigma)) and 1% (final concentration) of alum. The fine dust mixture was directly injected into the airways and noses of the experimental animals on days 4, 7, and 10 of the experiment. (Lim et al., Free Radic Biol Med. 25 (6), 635-644 (1998), Shin et al., Korean J. Mediclinal Crop Sci 27 (3), 218-231 (2019)). A positive control was administered orally daily for 10 days, dexamethasone (dexamethasone, also known as Dexa, Sigma, D2915) at a concentration of 3 mg/kg BW, and Lactic acid bacterium Lactobacillus paracasei ATG-E1 at a concentration of 4×10⁹ CFU/mouse diluted in 0.5% sodium carboxymethyl cellulose solution (CMC, Sigma, 419273). An autopsy was performed on the 11th day after the start of the experiment, and bronchoalveloar lavage fluid (BALF) and lung tissue were collected.

### Example 3-2 Measurement of Total Cell Count and Neutrophil Count in Bronchoalveolar Lavage Fluid (BALF)

On the last day of the experiment in Example 3-1, bronchoalveolar lavage fluid (BALF) was obtained after anesthesia, stained with 0.04% trypan blue, and the total number of cells was calculated using a hematocytometer to determine the total number of cells in the bronchoalveolar lavage fluid. Next, the sample was smeared with cytosine and stained with Diff-Qick, and the number of neutrophils was differentially calculated through optical microscopy.

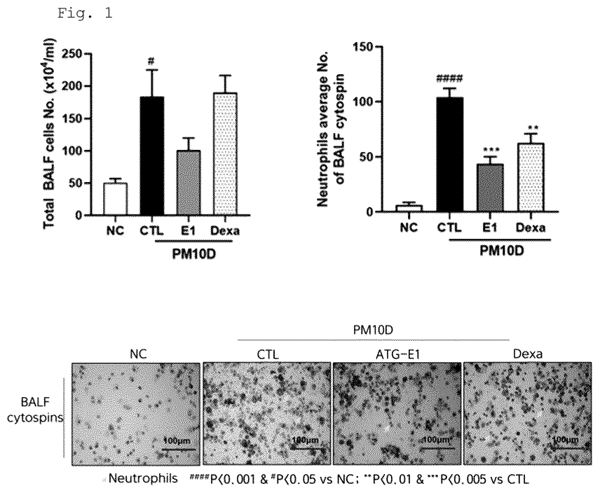

As a result, the total number of cells and the number of neutrophils, which are inflammatory immune cells, in the bronchoalveolar lavage fluid were significantly increased in the fine dust-induced respiratory injury group (CTL), and the total number of cells in the bronchoalveolar lavage fluid tended to decrease in the group treated with Lactobacillus paracasei ATG-E1 (ATG-E1) after the respiratory injury was induced with the fine dust mixture. In addition, the number of neutrophils cells increased by the fine dust mixture (air pollutant) was significantly reduced, as shown in FIG. 1.

The results show that the administration of Lactobacillus paracasei ATG-E1 ameliorates the respiratory disease induced by the fine dust.

### Example 3-3. Analysis of all Immune Cells in Bronchoalveolar Lavage Fluid (BALF)

Immune cell profiles (lymphocytes, neutrophils, macrophages, T cell subtypes, Gr-1⁺CD11b⁺, etc.) were measured by fluorescence-activated cell sorting (FACS) analysis of bronchoalveolar lavage fluid isolated from each individual after the end of the experiment in Example 3-1.

TABLE 6

| | | | PM10D-induced airway inflammation model | | |
|---|---|---|---|---|---|
| | | | (Absolute No.) | | |
| Cell phenotype | | NC | CTL | ATG-E1 | Dexa_3 mg/kg |
| Lymphocytes (×10⁷ | BAL | 0.08 ± 0.02 | 0.82 ± 0.05^{###} | 0.87 ± 0.25 | 0.96 ± 0.53 |
| cells) | | | | | |
| Neutrophils (×10⁷ | | 3.59 ± 0.73 | 15.09 ± 3.49^{##} | 7.58 ± 1.40* | 15.01 ± 2.14 |
| cells) | | | | | |
| Macrophage (×10⁷ | | 1.18 ± 0.14 | 1.82 ± 0.49 | 1.25 ± 0.15 | 2.29 ± 0.28 |
| cells) | | | | | |
| CD4⁺ (×10⁷ cells) | | 0.07 ± 0.03 | 5.26 ± 1.14^{###} | 2.18 ± 0.48** | 3.43 ± 0.58 |
| CD8⁺ (×10⁷ cells) | | 0.14 ± 0.08 | 3.72 ± 0.52^{###} | 2.54 ± 0.52 | 2.80 ± 0.71 |
| CD4⁺CD69⁺ (×10⁷ cells) | | 0.08 ± 0.04 | 1.73 ± 0.52^{##} | 0.61 ± 0.10* | 1.40 ± 0.19 |
| CD62L⁻CD44^{+high} | | 0.99 ± 0.21 | 6.40 ± 1.52^{##} | 2.54 ± 0.41* | 5.95 ± 0.85 |
| (×10⁷ cells) | | | | | |
| Gr-1⁺CD11b⁺ (×10⁷ | | 0.34 ± 0.06 | 1.62 ± 0.42^{##} | 0.79 ± 0.14 | 1.38 ± 0.12 |
| cells) | | | | | |

^{##}P < 0.01 & ^{###}P < 0.005 vs NC;
*P < 0.05 vs CTL

The number of each type of immune cells is shown in Table 6. In the CTL group in which the respiratory injury was induced with the fine dust mixture, the number of all immune cells was significantly increased. However, in the ATG-E1 group to which Lactobacillus paracasei ATG-E1 was administered after the respiratory injury was induced with the fine dust mixture, the number of neutrophils, CD4⁺, and CD4⁺CD69⁺, which are immune cells, and the number of CD62L-CD44^{+high} cells, which are specific markers for inflammatory response induced by the fine dust mixture, were significantly reduced.

### Example 3-4. Analysis of Immune Cells in Cells Isolated from Lung Tissue

Immune cell profiles (lymphocytes, neutrophils, macrophages, T cell subtypes, Gr-1⁺CD11b⁺, etc.) were measured by fluorescence-activated cell sorting (FACS) analysis of bronchoalveolar lavage fluid isolated from lung tissue of each individual after the end of the experiment in Example 3-1.

TABLE 7

| | | | PM10D-induced airway inflammation model | | |
|---|---|---|---|---|---|
| Cell phenotype | | | (Absolute No.) | | |
| (FACS analysis) | | NC | CTL | ATG-E1 | Dexa_3 mg/kg |
| Lymphocytes (×10⁷ | Lung | 2.60 ± 0.64 | 3.17 ± 0.51 | 4.39 ± 0.83 | 9.34 ± 6.56 |
| cells) | | | | | |
| Neutrophils (×10⁷ | | 3.03 ± 0.48 | 12.04 ± 2.26^{##} | 5.06 ± 0.67* | 3.41 ± 0.83** |
| cells) | | | | | |
| Eosinophils | | 1.56 ± 0.20 | 6.87 ± 0.97^{###} | 4.28 ± 1.02 | 3.41 ± 1.06 |
| (×10⁷ cells) | | | | | |
| CD4⁺ (×10⁷cells) | | 1.97 ± 0.35 | 6.13 ± 1.08^{##} | 3.39 ± 0.62* | 3.32 ± 0.42* |
| CD8⁺ (×10⁷ cells) | | 1.11 ± 0.23 | 3.46 ± 0.78^{##} | 1.84 ± 0.28 | 1.92 ± 0.18 |
| CD4⁺CD69⁺ (×10⁷ cells) | | 0.20 ± 0.08 | 3.01 ± 1.08* | 0.46 ± 0.11* | 0.29 ± 0.07* |
| CD62L⁻CD44^{+high} (×10⁷ | | 0.35 ± 0.05 | 2.53 ± 0.50^{##} | 0.87 ± 0.06* | 1.27 ± 0.15 |
| cells) | | | | | |
| CD21/35 + B220⁺ (×10⁷ | | 0.27 ± 0.09 | 3.40 ± 0.72^{###} | 1.81 ± 0.35* | 1.35 ± 0.32** |
| cells) | | | | | |
| Gr-1⁺CD11b⁺ (×10⁷cells) | | 0.17 ± 0.05 | 1.07 ± 0.09^{###} | 0.38 ± 0.06*** | 0.60 ± 0.08*** |

^{##}P < 0.01 & ^{###}P < 0.005 vs NC;
*P < 0.05 &
****P < 0.0005vs CTL

The results are shown in Table 7 below. The results showed that in the lung tissue, the number of all immune cells was significantly increased in the fine dust-induced respiratory injury group CTL, while the number of neutrophils, CD4+, CD4⁺CD69⁺, and CD62L-CD44^{+high}, CD21/35⁺B220⁺, and Gr-1⁺CD11b⁺ cells, which are specific markers for the fine dust-induced inflammatory response, were significantly reduced in the ATG-E1 group treated with Lactobacillus paracasei ATG-E1 after the respiratory injury was induced by the fine dust mixture.

### Example 3-5. Measurement of Production of Total Inflammatory Factors in Bronchoalveolar Lavage Fluid (BALF)

The expression levels of inflammatory factors such as interleukin-17A (IL-17A), tumor necrosis factor-α (TNF-α), macrophage inflammatory protein 2 (MIP2), and C-X-C motif chemokine ligand 1 (CXCL-1) in the bronchoalveolar lavage fluid (BALF) of each of the individuals used in the experiment of Example 3-1 was measured with a commercially available ELISA kit (R&D system, USA) according to the test method provided by the manufacturer.

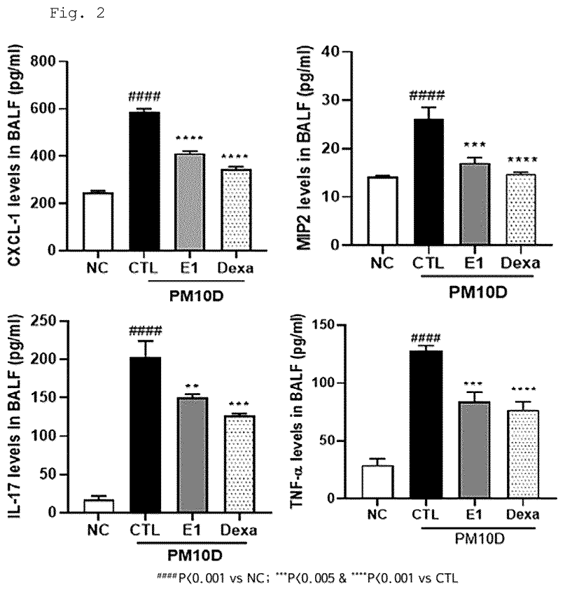

As shown in FIG. 2, the production levels of the inflammatory factors such as IL-17A, TNF-α, MIP2, and CXCL-1 were significantly increased in the CTL group with respiratory injury induced by the fine dust mixture, while the production levels of the inflammatory factors were significantly reduced in the ATG-E1 group because the mice in the group were treated with Lactobacillus paracasei ATG-E1 after suffering the respiratory injury induced by the fine dust mixture.

### Example 3-6. Expression Analysis of Inflammatory Factors in Lung Tissue

Total RNA was extracted using RNAzol B reagent (Tel-Test, Austin, TX, USA) from the excised lung tissue of each of the individuals in the experiment of Example 3-1, and 3 μg of the total RNA was used to synthesize cDNA using ReverTraAce-a-cDNA Synthesis kit (Toyobo, Osaka, Japan). The synthesized DNA was subjected to real-time polymerase chain reaction (real-time PCR) using an Applied Biosystems 7500 Real-time PCR system (Applied Biosystems, USA) to analyze the expression levels of interleukin-6 (IL-6), TNF-α, CXCL-1, and MIP-1a. The real-time PCR was performed under such conditions that pre-denaturation was performed at 50° C. for 2 minutes, followed by heating at 94° C. for 10 minutes, followed by 40 cycles of treatment at 94° C. for 1 minute and 60° C. for 1 minute. The primer sequences used for genetic analysis are shown in Table 8. For the sample-administered group and the control group, the relative quantitative (RQ) was measured using GAPDH as an internal standard.

TABLE 8

| Gene | Sequence (5′-3′) |
|---|---|
| IL-6 | Forward: TCCAGTTGCCTTCTTGGGAC |
| | Reverse: GTGTAATTAAGCCTCCGACTTG |
| TNF-α | Forward: GGCTTTCCGAATTCACTGGAGCCT |
| | Reverse: CCCCGGCCTTCCAAATAAATACATTCATA |
| MIP-1α | Forward: CACCATATGGCTCGGACACC |
| | Reverse: TCAGGAAAATGACACCTGGCT |
| CXCL1 | Forward: CCGAAGTCATAGCCACAC |
| | Reverse: GTGCCATCAGAGCAGTCT |
| GAPDH | Forward: GTCTTCCTGGGCAAGCAGTA |
| | Reverse: CTGGACAGAAACCCCACTTC |

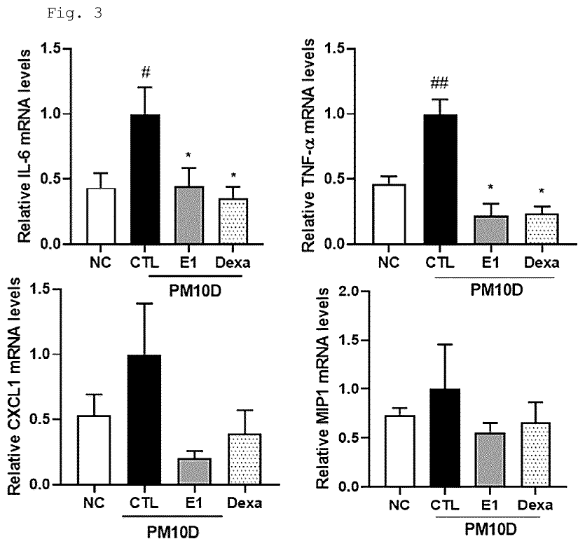

The real-time PCR results are shown in FIG. 3. The results show that the expression of the inflammatory cytokines “IL-6, TNF-α, CXCL-1, and MIP-1α” was increased in the lung tissue of the fine dust-induced respiratory injury group CTL, but the gene expression of the inflammatory cytokines was significantly reduced by Lactobacillus paracasei ATG-E1 treatment.

### Example 3-7. Histopathological Examination

The lung tissue was harvested from each of the individuals used in the experiment of Example 3-1, fixed in 10% neutral buffered formalin, and embedded in paraffin to prepare blocks from which tissue sections (4 μm thick) were prepared. Hematoxylin & Eosin (H&E) staining was then performed to observe inflammation in the lung tissue. Masson's trichrome (MT) staining for collagen deposition staining, and periodic acid Schiff (PAS) staining for mucus secretion staining were performed to observe pathological changes in the lung tissue through optical microscopy.

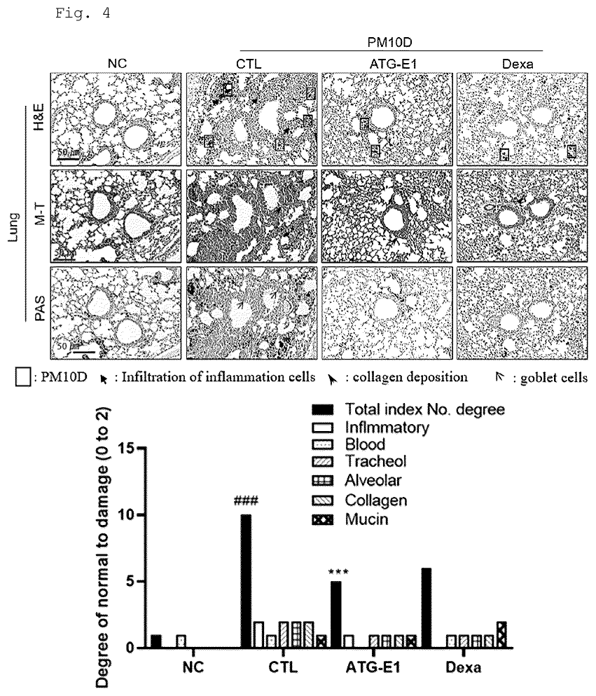

The observation results are shown in FIG. 4. The results show that the fine dust-induced respiratory injury group (CTL) had thickened lung cell walls, had increased collagen fibers, and exhibited inflammatory symptoms indicating tissue damage compared to the normal group, but the tissue damage was alleviated in the Lactobacillus paracasei ATG-E1 treatment group.

### Formulation Example 1. Pharmacological Formulations

### Formulation Example 1-1. <Preparation of Tablets>

200 g of Lactobacillus paracasei ATG-E1 of the present disclosure was mixed with 175.9 g of lactose, 180 g of potato starch, and 32 g of colloidal silicic acid. A 10% gelatin solution was added to the mixture, and the mixture was pulverized and passed through a 14-mesh sieve. The residue was dried and added with 160 g of potato starch, 50 g of talc, and 5 g of magnesium stearate, and the mixture was tableted.

### Formulation Example 2. Preparation of Food

### Formulation Example 2-1. Preparation of cooking seasonings

A health functional cooking seasoning was prepared by adding 1 wt % of Lactobacillus paracasei ATG-E1 of the present disclosure to a cooking seasoning.

### Formulation Example 2-2. Preparation of Dairy Products

Various dairy products such as butter and ice cream were prepared by adding 0.1 wt % of Lactobacillus paracasei ATG-E1 of the present disclosure to milk.

### Formulation Example 2-5. Preparation of Vegetable Juice

A health functional vegetable juice was prepared by adding 0.5 g of Lactobacillus paracasei ATG-E1 of the present disclosure to 1,000 ml of tomato juice or carrot juice.

### Formulation Example 2-6. Preparation of Fruit Juice

A health functional fruit juice was prepared by adding 0.1 g of Lactobacillus paracasei ATG-E1 of the present disclosure to 1,000 ml of apple juice or grape juice.

### Depository Authority

Name of Depository Authority: Korean Collection for Type Cultures, Korea Institute of Bioscience and Biotechnology
Accession Number: KCTC 14245BP
Date of Deposit: Jun. 21, 2020

### Budapest Treaty on the International Recognition of the Deposit of Microorganisms for the Purposes of Patent Procedure

INTERNATIONAL FORM RECEIPT FOR ORIGINAL DEPOSIT issued pursuant to Rule 7.1

### NAME OF DEPOSITOR: Jihee GANG

ADDRESS: Jihee GANG, 11-8, Techno 1-ro, Yuseong-gu, Daejeon, Republic of Korea

1. Lactobacillus paracasei ATG-E1 strain having the accession number of KCTC 14245BP.

2. The Lactobacillus paracasei ATG-E1 strain of claim 1, wherein the strain comprises a live cell thereof, a killed cell thereof, a culture thereof, a culture fluid comprising a cell thereof, a culture fluid from which a cell thereof has been removed, a concentrate of a culture fluid thereof, and a metabolite isolated from the cell or culture fluid.

3-12. (canceled)

13. A method for preventing or treating a respiratory disease, comprising administering to a subject in need thereof the Lactobacillus paracasei ATG-E1 strain according to claim 1.

14. The method according to claim 13, wherein the strain comprises a live cell thereof, a killed cell thereof, a culture thereof, a culture fluid comprising a cell thereof, a culture fluid from which a cell thereof has been removed, a concentrate of a culture fluid thereof, and a metabolite isolated from the cell or culture fluid.

15. The method according to claim 13, wherein the respiratory disease comprises a disease selected from the group consisting of acute and chronic bronchitis, catarrhal bronchitis, bronchitis obliterans, inflammatory bronchitis, bronchial asthma, atopic asthma, non-atopic asthma, atopic IgE-mediated asthma, allergic asthma, non-allergic asthma, chronic bronchoconstriction, acute bronchoconstriction, chronic obstructive pulmonary disease, bronchial adenoma, pulmonary tuberculosis, emphysema, lung abscess, pulmonary fibrosis, lung cancer, airway cancer, bronchoalveolar cancer, and bronchial cancer.

16. The method according to claim 13, wherein the subject requires reducing a number of inflammatory cells and immunoregulatory cells in a bronchoalveolar or lung tissue.

17. The method according to claim 13, wherein the subject requires reducing expression of inflammatory cytokines including interleukin-17A (IL-17A), tumor necrosis factor-α (TNF-α), macrophage inflammatory protein 2 (MIP2), C-X-C motif chemokine ligand 1 (CXCL-1), macrophage inflammatory protein-1a (MIP-1α) or interleukin-6 (IL-6).
